SEQUENCE LISTING (6 sequences)

SEQ ID NO: 1 - DNA, 1769 bp, Homo sapiens
gcccacgtctggttcaggggctagaaaagagcgtcgatgccggcggcagtgatgagtcctaggaggcgctggctctttggcggctcggaggagcggctgctgctgctgctgctgctgctggtggcccctttgcagatgtattgctgtccttgaatattagcccatttgaaaacgcctgggaagttcagccatcagtatgtccaagtacaaacttattatgttaagacatggagagggtgcttggaataaggagaaccgtttttgtagctgggtggatcagaaactcaacagcgaaggaatggaggaagctcggaactgtgggaagcaactcaaagcgttaaactttgagtttgatcttgtattcacatctgtccttaatcggtccattcacacagcctggctgatcctggaagagctaggccaggaatgggtgcctgtggaaagctcctggcgtctaaatgagcgtcactatggggccttgatcggtctcaacagggagcagatggctttgaatcatggtgaagaacaagtgaggctctggagaagaagctacaatgtaaccccgcctcccattgaggagtctcatccttactaccaagaaatctacaacgaccggaggtataaagtatgcgatgtgcccttggatcaactgccacggtcggaaagcttaaaggatgttctggagagactccttccctattggaatgaaaggattgctcccgaagtattacgtggcaaaaccattctgatatctgctcatggaaatagcagtagggcactcctaaaacacctggaaggtatctcagatgaagacatcatcaacattactcttcctactggagtccccattcttctggaattggatgaaaacctgcgtgctgttgggcctcatcagttcctgggtgaccaagaggcgatccaagcagccattaagaaagtagaagatcaaggaaaagtgaaacaagctaaaaaatagtctttctcaactgttggctaagaagaaatgcaaaagaagtggcataggagtgtgttatgggtgctgaactctctctctttttccccgattttccagagctaggctgtggagtagagtttgtataggtaactaggtaacttattgtggcccagataaggctttaggatgcctcagtgcttatgtcatagccttatgagttagctttcttgctagccccctagtcggtcaccaaactagtaactagtggggcttaatgaaggtcataagtttctgagatgggagagcaacaagtagagatgaagttaaaggtatttatcattcaagaaatcattattgagtcaccattgacaggcactattctaatcagtagttcactttaatatttaataagattttctgggataacagtaagggatattagataatataccgtatgtatttattactagtcttttcctctaggaaaagggatactttgataattaaggccagaggcccattagttgagaaagtcacagatatatttctccaagaaagccaacaaccaccaccacaatgacagaaatgacaacaaggccctttaacttgtcttctagtttagagacatccttcatttgacatttagtagaattcctctttggccacaagaataagcagcaaataaacaactatggctgttgaggttctcattttggtttgttttaattttttgaactttgggtacctgtaattagtttaaaaataaagttcctgataataaagtgactgaaaatggcatccccaaaaaaaaaaa

SEQ ID NO: 2 - protein, 259 aa, Homo sapiens
MSKYKLIMLRHGEGAWNKENRFCSWVDQKLNSEGMEEARNCGKQLKALNFEFDLVFTSVLNRSIHTAWLILEELGQEWVPVESSWRLNERHYGALIGLNREQMALNHGEEQVRLWRRSYNVTPPPIEESHPYYQEIYNDRRYKVCDVPLDQLPRSESLKDVLERLLPYWNERIAPEVLRGKTILISAHGNSSRALLKHLEGISDEDIINITLPTGVPILLELDENLRAVGPHQFLGDQEAIQAAIKKVEDQGKVKQAKK

SEQ ID NO: 3 - DNA, 16 bp, Artificial
  oligonucleotide primer
tggtggcccctttgca

SEQ ID NO: 4 - DNA, 20 bp, Artificial
  oligonucleotide primer
ggcgttttcaaatgggctaa

SEQ ID NO: 5 - DNA, 20 bp, Artificial
  probe specific for BPGM encoding nucleic acid (human)
agccaacagttgagaaagac

SEQ ID NO: 6 - DNA, 26 bp, Artificial
  probe specific for BPGM encoding nucleic acid (mouse)
ataaactgctaccactgtgccatacc

(19) United States
(12) Patent Application Publication
(10) Pub. No.: US 2008/0261213 A1
(43) Pub. Date: Oct. 23, 2008

(54) Assay

(75) Inventor: Manu Vatish, Coventry (GB)

Correspondence Address: BARNES & THORNBURG LLP, 11 SOUTH MERIDIAN, INDIANAPOLIS, IN, 46204, US

(73) Assignee: UNIVERSITY OF WARWICK, Coventry (GB)

(21) Appl. No.: 11/721,646

(22) PCT Filed: Dec. 16, 2005
(86) PCT No.: PCT/GB05/04901

§ 371 Date: Oct. 23, 2007

(30) Foreign Application Priority Data
Dec. 16, 2004 (GB) 0427578.0

(51) Int. Cl. C12Q 1/68; C12Q 1/48; G01N 33/573; C12Q 1/02

(52) U.S. Cl. 435/6; 435/15; 435/7.4; 435/29

(57) ABSTRACT

The invention relates to a method for screening for pre-eclampsia in a mammal, such as a human, by determining the amount and quality of foetal 2,3 bisphosphoglycerate mutase (2,3 BPGM) present. Reagents and kits for carrying out the method are also provided.

The invention relates to a method for screening for pre-eclampsia in a mammal, such as a human, by determining the amount and quality of foetal 2,3 bisphosphoglycerate mutase (2,3 BPGM) present. Reagents and kits for carrying out the method are also provided.

Pre-eclampsia is a disorder unique to pregnancy (affecting about 10% of pregnancies), characterised by high blood pressure i.e. blood pressure of >140/90 mm Hg (on at least two occasions 6 hours apart) and the presence of protein in the urine. In some cases (1-2% of pregnancies), convulsions or coma or both may develop resulting in eclampsia. It endangers both the mother and foetus and along with other hypertensive pregnancy disorders, is one of the main causes of maternal and perinatal morbidity and mortality. In the developed world, pre-eclampsia is estimated to play a role in almost 1 out of every 5 maternal deaths and accounts for some 15% of premature births. The costs associated with managing pre-eclampsia have been estimated to be in the region of 10 billion US dollars per year with a similar figure being suggested in coping for disease after birth resulting from pre-eclampsia during pregnancy. These later effects include the psychological and physical effects on the affected mother (cerebral haemorrhage and adult respiratory distress syndrome) and many infant conditions associated with premature birth and intrauterine growth restriction due to pre-eclampsia, ranging from respiratory distress in premature babies to cerebral palsy, blindness, epilepsy, deafness, and learning disabilities. In severe cases, intrauterine death may occur. The detrimental effects of pre-eclampsia upon the health of women and children all over the world has prompted the World Health Organisation to launch a global program to combat this disorder.

Pre-eclampsia is a rapidly progressive disorder affecting multiple organ systems. In severe cases, a multidisciplinary approach in an intensive care setting is absolutely crucial in the successful care of these patients. The current management of pre-eclamptic patients concentrates upon intensive maternal and foetal surveillance employing a wide range of blood tests, urinalysis and ultrasonography (Doppler). The use of anti-hypertensives (methyldopa, nifedipine, hydralazine and labetalol) is well recognised, particularly in the prevention of cerebro-vascular accidents. The ultimate treatment of pre-eclampsia is the delivery of the placenta (and the baby) that invariably abates the progression of this disease.

The pathophysiology of pre-eclampsia has been well studied. The underlying abnormality is generalised vasoconstriction of the arterioles and enhanced sensitivity of these blood vessels to vasopressor peptides and amines. It has been proposed by some investigators that an imbalance of prostaglandins i.e. Prostacyclin (vasodilator and platelet aggregator inhibitor) and Thromboxane A2 (vasoconstrictor and platelet aggregator) is central in the development of pre-eclampsia. Other investigators have found that there is decreased production of nitric oxide, an endogenous vasodilator, in pre-eclampsia. However, despite extensive research, the exact aetiology of pre-eclampsia remains an enigma. In relation to this, the placenta has been identified as the organ with a pivotal role in the pathogenesis of pre-eclampsia. Essentially, in pre-eclampsia, placentation and trophoblast invasion is abnormal. This compromises the utero-placental circulation and results in placental ischaemia.

### Pre-Eclampsia Assays are Known.

WO 91/16633 shows a pre-eclampsia marker based on A134-binding cell marker. This is assayed using an anti-(cellular fibronectin) antibody.

U.S. Pat. No. 5,198,366 discloses pp-13 as a marker for pre-eclampsia, intra-uterine growth retardation and pre-term delivery.

Cytokines have been implicated in pre-eclampsia. Hence, M-CSF levels have been suggested as an assay target and therapeutic agent (U.S. Pat. No. 5,543,138).

Other markers that have been tried to be assayed include Insulin-like Growth Factor Binding Protein 1 (U.S. Pat. No. 5,712,103), Marinobutagenin (WO 2004/071273), defensins (WO 99/42826) and free albumin: non-esterified fatty acid ratios (WO 01/77675). Mitogens have also been assayed (U.S. Pat. No. 5,238,819), as have phosphatidyl choline (U.S. Pat. No. 6,461,830).

Syncytin levels have been used as targets for pre-eclampsia drugs (WO 02/04678 and US 2002/0102530).

U.S. Pat. No. 5,849,474 discloses an assay method which looks for haemoglobin variants, haemoglobin variant precursors or red blood cell glycolytic enzymes or precursors of such enzymes from the blood of a pregnant female mammal. The assayed compounds are produced within the female mammal's red blood cells. The document suggests that reduced levels of 2,3 diphosphoglyceric acid (2,3-DPG) could indicate an interruption in glycolysis, resulting in decreased ATP production and increased haemolysis. 2,3-DPG increase in normal pregnancy causes a shift in the oxyhaemoglobin dissociation curve for the mother's blood which increases the supply of oxygen made available to not only maternal tissues, but also for transport to the foetus.

There is a complicated enzymatic cascade effecting the production of 2,3-DPG involving a large number of different enzymes. U.S. Pat. No. 5,849,474 suggested that one or more of these enzymes from the mother might be involved in affecting 2,3-DPG levels. A problem with this approach is that there are a number of different factors which affect the 2,3-DPG production in mothers, including stress factors as diverse as the altitude at which the mother lives and whether the mother smokes.

Although there are several different pre-eclampsia assays known, there is still a need to produce improved assays for determining the presence of the condition as early as possible during pregnancy, to allow treatment of the condition before further harm is done to either the mother or the foetus.

The inventors used a mouse model in the initial investigation into this condition. The Igf2 knockout mouse produces small pups that suffer similar growth restriction to that seen in babies of human mothers suffering pre-eclampsia (Constancia, M., et al., Nature (2002); 417; 945-948).

Mouse placentae were screened for gene expression using a commercially available expression array assay. This identified some 300 putative candidate genes associated with this growth restriction. From these candidate genes the inventors identified the 2,3 bisphosphoglycerate mutase (2,3 BPGM) gene as a candidate for studying further. The presence of this enzyme was confirmed in both human and mouse placentae by real-time PCR. Moreover, in situ hybridisation indicates abundant mRNA for 2,3 BPGM in the syncytiotrophoblast layer of placental villi in both human and mouse placentae.

2,3 BPGM catalyses the conversion of 1,3-bisphosphoglycerate into 2,3 bisphosphoglycerate (2,3 BPG). 2,3 BPG was previously called 2,3 diphosphoglycerate, hence 2,3 BPGM is also known as 2,3 diphosphoglycerate mutase (DPGM). This enzyme has been characterised and sequenced, as indeed shown in the following papers:
Wang, Y., Wei, Z., Bian, Q., Cheng, Z., Wan, M., Liu, L. and Gong, W. Crystal structure of human bisphosphoglycerate mutase. J. Biol. Chem. (2004); 279(37); 39132-38.
Joulin, V., Garel, M. C., Le Boulch, P., Valentin, C., Rosa, R., Rosa, J. and Cohen-Solal, M. Isolation and characterization of the human 2,3-bisphosphoglyceerate mutase gene. J. Biol. Chem. (1988); 263(30); 15785-90.
Barichard, F., Joulin, V., Henry, I., Garel, M. C., Valentin, C., Rosa, R., Cohen-Solal, M. and Junien, C. Chromosomal assignment of the human 2,3-bisphosphoglycerate mutase gene (BPGM) to region 7q34 - - - 7q22. Hum. Genet. (1987); 77(3); 283-285.
Joulin, V., Peduzzi, J., Romeo, P. H., Rosa, R., Valentin, C., Dubart, A., Lapeyre, B., Blouquit, Y., Garel, M. C., Goossens, M., et al. Molecular cloning and sequencing of the human erythrocyte 2,3-bisphosphoglycerate mutase cDNA: revised amino acid sequence. EMBO J. (1986); 5(9); 2275-83.

Previously, this enzyme was known to be associated with maternal red blood cells. This has not previously been observed in the placentae of mammals. The enzyme is synthesised in the syncytiotrophoblast layer of the placenta. This layer is the outer-most layer of cells that sheath the placental villi and are in direct contact with the maternal blood supply. That is, the enzyme is expressed at the interface between the maternal and foetal circulation. The presence of 2,3 BPGM in the placenta, along with its only ascribed role being the modulation of oxygen released from haemoglobin, with a differential effect on foetal and maternal haemoglobin, indicates that this protein may be key in some disorders of pregnancy, such as pre-eclampsia. Fragments of the syncytiotrophoblast layer are known to be released into the maternal blood supply. Hence, 2,3 BPGM produced by the foetus can be detected within samples of bodily fluid and tissues obtained from the mother.

Accordingly, the invention provides:
a method for screening for placental insufficiency, such as pre-eclampsia in a pregnant mammal such as a human or mouse comprising the steps of:
(i) providing a sample of maternal or foetal bodily fluid or tissue;
(ii) measuring the amount of foetal 2,3 bisphosphoglycerate mutase (2,3 BPGM) or a variant thereof in the sample; and
(iii) comparing the amount with a known normal level of foetal 2,3 BPGM or a variant thereof.

The invention also provides
a method for screening for placental insufficiency, such as pre-eclampsia in a pregnant mammal such as a human or mouse comprising the steps of:
(i) providing a sample of maternal bodily fluid or tissue;
(ii) detecting the presence of a foetal 2,3 BPGM or variant thereof in the sample

The invention also provides

a method for screening for placental insufficiency, such as pre-eclampsia in a pregnant mammal such as a human or mouse comprising the steps of:
(i) providing a sample of maternal or foetal bodily fluid or tissue;
(ii) measuring the amount of foetal 2,3 bisphosphoglycerate mutase (2,3 BPGM) or a variant thereof, and/or detecting the presence of a foetal 2,3 BPGM or variant thereof in the sample; and
(iii) comparing the amount with a known normal level of foetal 2,3 BPGM or a known variant thereof.

Preferably, the placental insufficiency is pre-eclampsia. However, it is expected that the enzyme may be involved in other forms of conditions where there is a problem with the placenta, such as intrauterine growth restriction.

The amount of foetal 2,3 BPGM is compared with known levels of 2,3 BPGM obtained from previously characterised pregnant mammals with, for example, normal pregnancies or with pre-characterised conditions such as pre-eclampsia. This allows an indication of the presence of the placental insufficiency to be determined. Further diagnostic methods, such as ultrasound of foetal growth may be used to confirm this insufficiency.

Preferably, the sample is selected from maternal blood, foetal blood, maternal urine, maternal faeces, maternal sputum, amniotic fluid and placental material such as a chorionic villus sample.

Preferably, it is maternal bodily fluid, such as blood, that is provided as the sample. Maternal blood has the advantage that it is relatively safely and easily obtained. Placental material may be isolated from the blood using techniques known in the art such as those disclosed in, for example:
Vona, G., Beroud, C., Benachi, A., Quenette, A., Bonnefont, J. P., Romana, S., Dumez, Y., Lacour, B., Paterlini-Brechot, P. Enrichment, immunomorphological, and genetic characterization of fetal cells circulating in maternal blood. Am. J. Pathol. (2002 January); 160(1); 51-8.
Levine, R. J., Qian, C., Leshane, E. S., Yu, K. F., England, L. J., Schisterman, E. F., Wataganara, T., Romero, R., Bianchi, D. W. Two-stage elevation of cell-free fetal DNA in maternal sera before onset of pre-eclampsia. Am. J. Obstet. Gynecol. (2004 March); 190(3); 707-13.

The amount, such as concentration or activity, of 2,3 BPGM may be determined as the concentration of the enzyme present or alternatively by measuring the level of enzymatic activity. Alternatively, or additionally, the concentration of, for example, mRNA encoding 2,3 BPGM may be measured. It is believed that variations in 2,3 BPGM may be due to differences in levels of expression of 2,3 BPGM or alternatively due to differences in the activity of 2,3 BPGM produced by the foetus.

The concentration of BPGM and its activity may be assayed by techniques known in the art. For example, Takubo, T., et al. (Journal of Clinical Laboratory Analysis (1998); 12; 263-267) disclose a method of producing an enzyme-linked immunosorbent assay (ELISA) system for the determination of 2,3 BPGM in human erythrocytes using a polyclonal anti-BPGM antibody. The same assay may be used to determine foetal BPGM. Monoclonal or polyclonal antibodies against BPGM may be produced using techniques well-known in the art, for example using the method of Kohler and Milstein. The antibodies used may be, for example, antibodies of the classes IgG, IgM, IgA, IgD or IgE, binding fragments and hybrid derivatives of antibodies including, for example, Fab, and F(ab′)₂ fragments of antibodies. Such antibodies preferably preferentially bind to 2,3 BPGM to allow the enzyme to be identified. Preferably, such antibodies or fragments have less than 10%, preferably less than 5% cross-reactivity with other compounds.

Immunoassays use the preferential binding property of the antibodies to allow the identification of 2,3 BPGM. Immunoassay methods known in the art include competition assays, sandwich assays, agglomoration assays, precipitation assays, transistor bridge probe, particle sorting, light disturbing, light scattering and ultrasonic code immunoassays. Such immunoassays may use as labels, for example, radioisotopes, enzymes such as horseradish peroxidase, fluorogenic, chromogenic or chemiluminescent substances. Such assays themselves are well-known per se in the art as indeed shown in, for example, WO 91/16633 and U.S. Pat. No. 5,712,103, incorporated herein by reference.

Levels of 2,3 BPGM expression may be determined by, for example, measuring the concentration of messenger RNA for the foetal BPGM in the sample. Suitable methods for determining the concentration of the BPGM includes real-time polymerase chain reaction (RT-PCR). Real-time PCR allows the determination of the concentration of messenger RNA using a suitable primer specific for the mRNA the enzyme. The technique itself is well-known in the art per se. For example, Wittwer, C. T., et al. disclose monitoring of DNA amplification using a number of different techniques in the article in Biotechniques (1997); 22; 130-138. This paper includes discussion of the commercially available SYBR Green™ dye. See also EP 1179600, WO 97/46707, WO 97/46712 and WO 97/46714 which disclose RT-PCR.

The enzymatic activity of the 2,3 BPGM may be determined by, for example, assaying the conversion of 1,3 BPG to 2, 3 BPG. The formation of 2,3 BPG may be determined by techniques known in the art, including, for example, immunologically by using antibodies specific for 1,3 BPG or 2,3 BPG to identify the relative concentrations of those compounds.

A 2,3 BPGM variant is preferably encoded by a nucleic acid sequence or amino acid sequence which varies from a 2,3 BPGM sequence such as the sequence shown in FIG. 3a or FIG. 3b by the addition, deletion or substitution of 1, 2, 3, 4, 5, 6, 7, 8, 9, 10 or more nucleotides or amino acids.

For example, there is redundancy in the genetic code which allows different nucleotide sequences to encode the same amino acids. The table below shows the mRNA triplets and the amino acids they encode:

| | U | C | A | G | |
|---|---|---|---|---|---|
| U | 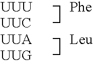 | 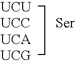 | 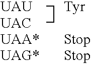 | 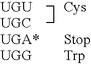 | 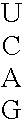 |
| C | 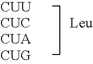 | 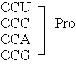 | 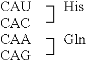 | 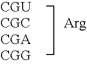 | 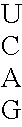 |
| A | 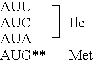 | 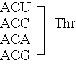 | 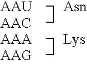 | 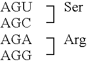 | 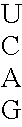 |
| G | 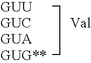 | 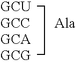 | 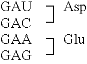 | 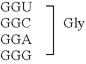 | 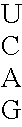 |

*Chain-terminating, or “nonsense” codons.
**Also used to specify the initiator formyl-Met-tRNAMet. The Val triplet GUG is therefore “ambiguous” in that it codes both valine and methionine.

It is known that variations in the nucleotide sequence encoding proteins or regulatory regions such as the promoter regions of genes encoding proteins may also affect the concentration of protein produced or the activity of the protein produced. Such variations, including single nucleotide polymorphisms (SNPs) are known to affect, for example, transcription levels of proteins or produce frame shift mutations or encode different amino acids. Such variations may result in the different levels of expression or activity observed. Hence, instead of or in addition to detecting the amount of foetal 2,3 BPGM variants may be detected. By variant, we mean the upstream region encoding the promoter or other regulatory region, such as enhancer region, as well as the region encoding the 2,3 BPGM protein (including exon and introns).

Preferably the variant has 2,3 BPGM activity. Preferably the variant is encoded by a nucleotide sequence which comprises a region complementary to or identical to a probe or primer according to the invention (defined below).

The inventors have also realised that because 2,3 BPGM seems to be important in identifying placental insufficiencies such as pre-eclampsia, then it may be possible to identify the level of a metabolite of 2,3 BPGM. Previously, it has been known that BPGM has been found within maternal red blood cells. However, the presence of the enzyme at the maternal blood-placental barrier would indicate that the enzyme produces a metabolite which, either directly or indirectly, diffuses into the blood to affect the oxygen dissociation curve of the maternal haemoglobin. Hence, a further aspect of the invention provides:

a method for screening placental insufficiency, such as pre-eclampsia in a pregnant mammal such as a human or mouse comprising the steps of:
(i) providing a sample of maternal or foetal bodily fluid or tissue;
(ii) measuring the concentration of free 2,3-bisphosphoglyceric acid (2,3 BPG); and
(iii) comparing the concentration with a known normal level of the 2,3-bisphosphoglyceric acid.

The concentration of free 2,3-bisphosphoglyceric acid is preferably the concentration outside foetal or maternal erythrocytes, for example in the blood plasma or interstitial fluid.

Methods of identifying and assaying for 2,3-bisphosphoglyceric acid are known in the art. However, as indicated previously, 2,3-bisphosphoglyceric acid may be assayed immunologically.

Preferably, the sample is selected from maternal blood, foetal blood, maternal urine, maternal faeces, maternal sputum, amniotic fluid and placental material such as a chorionic villus sample. The sample is preferably maternal blood, maternal urine or maternal sputum.

Preferably, a change in the concentration of the 2,3-bisphosphoglyceric acid or in the amount of 2,3 BPGM compared to a normal sample is indicative of a placental insufficiency.

The importance of 2,3 BPGM indicates that it is likely to be a target for the identification of drug candidates for the treatment of pre-eclampsia. Accordingly, a further aspect of the invention provides:

a method of determining a drug candidate for the treatment of placental insufficiency, such as pre-eclampsia comprising:
(i) providing a cell expressing foetal 2,3 BPGM;
(ii) introducing a compound to the cell;
(iii) determining the effect of the compound on the amount of 2,3 BPGM produced by the cell.

Preferably, the cell is a foetal cell, such as a foetal placental cell. The amount of 2,3 BPGM produced by the cell, mRNA encoding 2,3 BPGM, or the enzymatic activity of 2,3 BPGM in the cell may be measured and the effect of the compound on that amount may be determined to identify agonists or antagonist of 2,3 BPGM production. Methods used for determining the amount of 2,3 BPGM may be as indicated above.

Probes and primers for use in the claimed invention are also provided:

| | |
|---|---|
| 5′ tggtggcccctttgca | (SEQ ID NO. 3) |
| and | |
| 5′ ggcgttttcaaatgggctaa | (SEQ ID NO. 4) |
| Human probe: | |
| 5′ agccaacagttgagaaagac | (SEQ ID NO. 5) |
| Mouse probe: | |
| 5′ ataaactgctaccactgtgccatacc | (SEQ ID NO. 6) |

Assay kits for use in the methods of the invention are also provided. The kits may comprise detection means, such as antibodies or primers for detecting the amount of 2,3 BPGM in a sample and instructions for using the assay kit.

Furthermore, foetal placental debris is a feature of a number of different diseases, including trisomy 21 and intrauterine growth restriction. The presence of 2,3 BPGM in the foetal placental debris allows BPGM to be used as a marker for such debris. Placental debris may be identified using the techniques shown in the papers by Vonag, et al. (Am. J. Pathol. (2002); 116; 51-58) or Levine, R. J. (Am. J. Obstet. Gynecol. (2004); 190; 707-13) and the 2,3 BPGM can be used as a marker to confirm the presence of such material. 2,3 BPGM may be detected, for example, using polyclonal antibodies against 2,3 BPGM or alternatively monoclonal antibodies raised against the enzyme.

The invention also provides:
a method of identifying foetal placental debris in a pregnant mammal comprising:
(i) providing a sample of maternal bodily fluid or tissue;
(ii) isolating the foetal placental debris;
(iii) detecting 2,3 BPGM in the debris.

The invention will now be described by way of example only, with reference to the following figures:

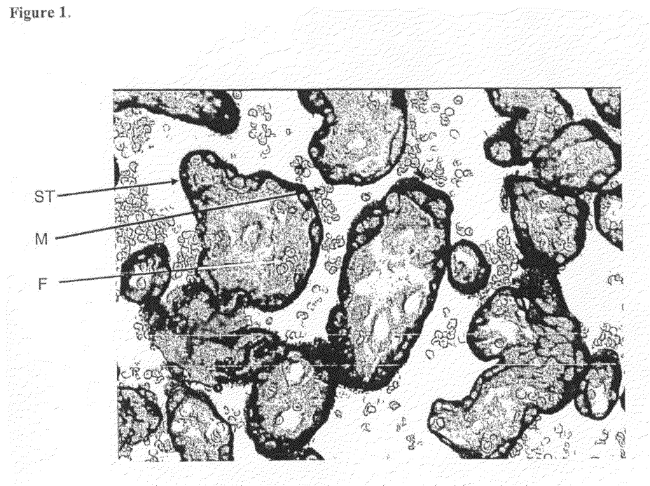

FIG. 1 shows in situ hybridisation probing for 2,3 BPGM in a transverse section of a placental villi. The 2,3 BPGM mRNA is found in the dark staining syncytiotrophoblast layer sheathing the villi (ST). Maternal erythrocytes (M) circulating between the villi and foetal erythrocytes (F) within the villi are indicated with arrows.

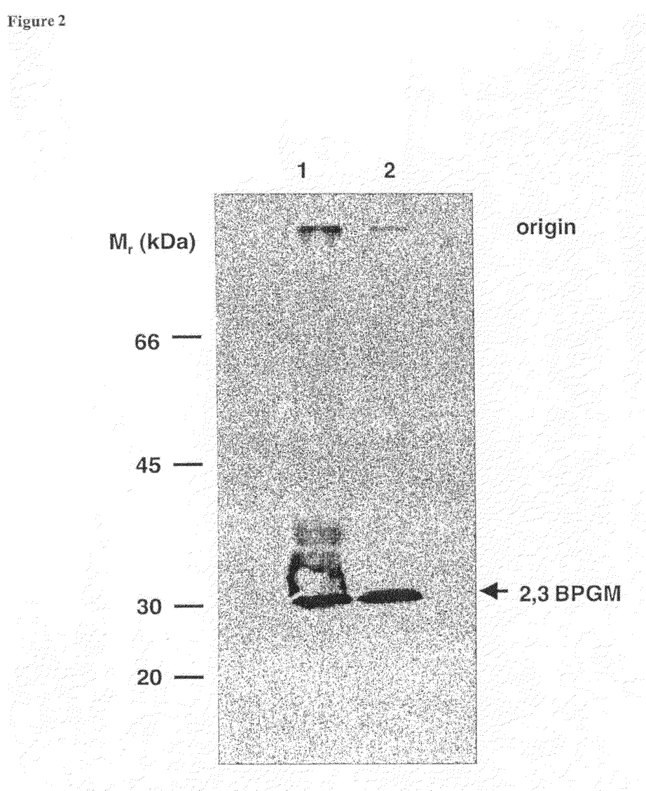

FIG. 2 shows a Western Blot analysis analysing SDS denatured proteins from adult human blood (lane 1) and placental tissue (lane 2) for the presence of 2,3 bisphosphoglycertate mutase. The band stained for 2,3 BPGM is indicated by the arrow.

FIG. 3 shows the nucleic acid (SEQ ID No. 1) and amino acid (SEQ ID No. 2) sequences for human 2,3 BPGM. The sequences of human 2,3 BPGM is also found at PubMed nucleotide entry NM_{—}199186 and, for mouse, as NM_{—}007563 and BC004589.

### MOUSE MODEL

Igf-2 knockout mouse were used as a model for growth restriction. These mice have small pups that suffer from similar growth restriction seen in human babies and mothers suffering from pre-eclampsia. The mouse model contains a deletion in the IgF2 gene.

Placentae from the Igf-2 mice were dissected. The expression of genes within the mouse placentae was analysed using an Affymetrix GeneChip™ mouse genome 430 2.0 array (Affymetrix UK Limited, High Wycombe, United Kingdom). This array contains an expression set that allows the analysis of the expression of over 39,000 transcripts and variants from over 34,000 mouse genes. MALDI-MS was carried out according to the manufacturer's instructions. Normalised expression values for wild-type mouse placental RNA samples was 1.463 (SD 0.125, N=3) indicating abundant expression. In the Igf-2 knockout mouse placental RNA expression was down-regulated to 0.563 (SD 0.086, N=3).

The data produced from the gene arrays was mined using GeneSpring software (Affymetrix Ltd.) which identified approximately 300 putative candidate genes associated with this growth restriction.

The inventors realised that human pregnancies affected with pre-eclampsia have more foetuses that are often at risk in utero death from lack of key nutrients, in particular oxygen. The inventors therefore focused on identifying genes involved in oxygen transfer and glycolysis.

The 2,3 BPGM gene showed three separate hits on the gene array data.

### Real-Time PCR

This was carried out on placental cDNA.

The mouse RT-PCR was carried out using a TaqMan™ gene expression assay purchased from Applied Biosystems Inc. The assay purchased was assay ID No. Mm 00500291_m1. This is an assay which allows the quantitation of 2,3-bisphosphoglycerate mutase in mice (Mus musculus).

The human RT-PCR was carried out on human placental cDNA using the following pnmers:
5′ tggtggcccctttgca (SEQ ID NO. 3) and 5′ ggcgttttcaaatgggctaa (SEQ II) NO. 4)

Real-time PCR was carried out using SYBR Green™ real-time PCR reagents and using the manufacturer's protocol (Applied Biosystems Inc.).

The real-time PCR confirmed that 2,3 BPGM is produced in the placentae of both humans and mice (data not shown).

In Situ Hybridisation Localisation of 2,3 BPGM mRNA in Human and Mice Placenta

Normal placentae were obtained from humans and mice.

Probes specific for BPGM encoding nucleic acid were prepared:

| | |
|---|---|
| Human probe: | |
| 5′ agccaacagttgagaaagac | (SEQ ID NO. 5) |
| Mouse probe: | |
| 5′ ataaactgctaccactgtgccatacc | (SEQ ID NO. 6) |

The probes were labelled and detected using a digoxigenin system using a commercially available kit and according to the manufacturer's instructions (Roche tailing kit, catalogue no. 3 353 583, Roche DIG nucleic acid detection kit, catalogue no. 1 175 041, available from F. Hoffmann-La Roche Ltd., United Kingdom).

Paraffin embedded sections were de-waxed in xylene and permeabilised using 0.3% Triton X100 in PBS for 15 minutes followed by treatment with 15 micrograms per mL proteinase K at 37° C. for 15 minutes. Probe hybridisation was at room temperature for 18 hours with 18% formamide, followed by two 15 minute washes in 2×SSC, two 15 min. washes in 1×SSC (standard saline citrate) and two 15 min. washes in 0.2×SSC. Bound probe was visualised using anti-DIG antibody conjugated to alkaline phosphatase developed using the NTB reagent supplied with the Roche kit and photomicrographs taken.

In both the mice and human sections the experiments showed that 2,3 BPGM mRNA is expressed in the syncytioptrophoblast layer of human placental villi, as indeed demonstrated in FIG. 1 for human placental villi.

### Immuno Localisation

Immuno localisation was carried out on human placental biopsy sections fixed in acetone, permeabilised in Triton X100™ and probed using mouse polyclonal antisera to human 2,3 BPGM. The mouse polyclonal antisera were prepared using standard methods in the art (see, for example, http://www.sigmaaldrich.com/Area_of Interest/Life_Science/Antibody_Explorer/Procedures/Immunofluorescence.html). Bound antibody was visualised with commercially available goat, anti-rabbit IgG fluorescently labelled with Texas Red (Vector Laboratories, Ca. USA). Fluorescence was observed in syncytiotrophoblast layers and in erythrocytes.

This data (not shown) confirmed that BPGM is found in the syncytiotrophoblast layer of the placenta and, as expected, in erythrocytes.

### Western Blot Analysis

Protein was extracted from human placental biopsies and denaturing SDS PAGE of the protein was carried out. This was electroblotted onto a nitrocellulose membrane along with a similarly fractionated human erythrocyte protein extract. Using standard techniques the membranes were blocked and probed using a polyclonal antisera specific for human 2,3 BPGM raised in rabbit. Examples of such polyclonal antibodies are shown, for example, in the article by Takubo, T. (Journal of Clinical Laboratory Analysis (1998); 12; 263-267).

The Western Blots were developed with an anti-rabbit-horseradish peroxidase secondary antibody and detected with ECL reagents using a commercially-available kit according to the manufacturer's instructions (Amersham Biosciences, product code no. RPN 2109).

FIG. 2 shows that a discreet band for 2,3 BPGM is found in both human blood and placental tissue.

The results show that 2,3 BPGM is unexpectedly found in placental tissue. This is the first time that the presence of this enzyme has been identified in this tissue. Furthermore, this enzyme is expressed in altered levels in mouse pre-eclampsia models and, thus indicating that it is a simple marker for the detection of placental insufficiency, such as pre-eclampsia.

Furthermore, foetal placental debris is a feature of a number of different diseases, including trisomy 21 and intrauterine growth restriction. The presence of 2,3 BPGM in the foetal placental debris allows BPGM to be used as a marker for such debris. Placental debris may be identified using the techniques shown in the papers by Vonag, et al. (Am. J. Pathol. (2002); 116; 51-58) or Levine, R. J. (Am. J. Obstet. Gynecol. (2004); 190; 707-13) and the 2,3 BPGM can be used as a marker to confirm the presence of such material. 2,3 BPGM may be detected, for example, using polyclonal antibodies against 2,3 BPGM or alternatively monoclonal antibodies raised against the enzyme.

1. A method for screening for placental insufficiency, such as pre-eclampsia in a pregnant mammal such as a human or mouse comprising the steps of:
(i) providing a sample of maternal or foetal bodily fluid or tissue;
(ii) measuring the amount of foetal 2,3 bisphosphoglycerate mutase (2,3 BPGM) or a variant thereof in the sample; and
(iii) comparing the amount with a known normal level of foetal 2,3 BPGM or a variant thereof.

2. A method for screening for placental insufficiency, such as pre-eclampsia in a pregnant mammal such as a human or mouse comprising the steps of:
(i) providing a sample of maternal bodily fluid or tissue;
(ii) detecting the presence of a foetal 2,3 BPGM or variant thereof in the sample.

3. A method according to claim 1, wherein the sample is selected from maternal blood, foetal blood, maternal urine, maternal faeces, maternal sputum, amniotic fluid and placental material such as a chorionic villus sample.

4. A method according to claim 2, wherein the sample is selected from maternal blood, maternal urine, maternal faeces, maternal sputum, amniotic fluid and placental material such as a chorionic villus sample.

5. A method according to claim 1, comprising the steps of providing a sample of maternal bodily fluid or tissue and measuring the amount of foetal 2,3 BPGM in the sample.

6. A method according to claim 5, wherein the sample is maternal blood, and the method comprises the steps of:
(a) isolating foetal placental material from the maternal blood; and
(b) measuring the amount of foetal 2,3 BPGM in the isolated foetal placental material.

7. A method according to claim 1, wherein the amount of foetal 2,3 BPGM is the concentration of 2,3 BPGM or mRNA encoding 2,3 BPGM in the sample or the level of 2,3 BPGM enzymatic activity in the sample.

8. A method according to claim 1, wherein the amount of 2,3 BPGM is determined by an immunoassay, enzymatic assay or real-time PCR.

9. A method for screening placental insufficiency, such as pre-eclampsia in a pregnant mammal such as a human or mouse comprising the steps of:
(i) providing a sample of maternal or foetal bodily fluid or tissue;
(ii) measuring the concentration of free 2,3-bisphosphoglyceric acid (2,3 BPG); and
(iii) comparing the concentration with a known normal level of the 2,3-bisphosphoglyceric acid.

10. A method according to claim 9, wherein the sample is selected from maternal blood, foetal blood, maternal urine, maternal faeces, maternal sputum, amniotic fluid and placental material such as a chorionic villus sample.

11. A method according to claim 10, wherein the sample is maternal blood, maternal urine or maternal sputum.

12. A method according to claim 9, wherein the 2,3-bisphosphoglyceric acid is measured by an immunoassay.

13. A method of determining a drug candidate for the treatment of placental insufficiency, such as pre-eclampsia comprising:
(i) providing a cell expressing foetal 2,3 BPGM;
(ii) introducing a compound to the cell;
(iii) determining the effect of the compound on the amount of 2,3 BPGM produced by the cell.

14. A method according to claim 13, wherein the cell is a foetal placental cell.

15. A method according to claim 13, wherein the amount of 2,3 BPGM is the level of 2,3 BPGM expression by the cell or the enzymatic activity of 2,3 BPGM.

16. An assay kit for use in a method according to claim 1 comprising detection means for measuring the amount of 2,3 BPGM in the sample and instructions for using the assay kit.

17. A method of identifying foetal placental debris in a pregnant mammal comprising:
(i) providing a sample of maternal bodily fluid or tissue;
(ii) isolating the foetal placental debris;
(iii) detecting 2,3 BPGM in the debris.

18. A method according to claim 2, comprising the steps of providing a sample of maternal bodily fluid or tissue and measuring the amount of foetal 2,3 BPGM in the sample.

19. An assay kit for use in a method according to claim 2, comprising detection means for measuring the amount of 2,3 BPGM in the sample and instructions for using the assay kit.

20. An assay kit for use in a method according to claim 9, comprising detection means for measuring the amount of 2,3 BPGM in the sample and instructions for using the assay kit.

21. An assay kit for use in a method according to claim 13, comprising detection means for measuring the amount of 2,3 BPGM in the sample and instructions for using the assay kit.
